(19) United States
(12) Patent Application Publication
(10) Pub. No.: US 2014/0378873 A1
(43) Pub. Date: Dec. 25, 2014

(54) METHODS, DEVICES AND SYSTEMS FOR INCREASING THE EFFECTIVENESS OF ULTRASOUND AND OTHER TISSUE TREATMENT MODALITIES

(71) Applicants: Ary S. Chernomorsky, Walnut Creek, CA (US); Michael Rontal, Farmington Hills, MI (US); Prasad Nalluri, Boerne, TX (US)

(72) Inventors: Ary S. Chernomorsky, Walnut Creek, CA (US); Michael Rontal, Farmington Hills, MI (US); Prasad Nalluri, Boerne, TX (US)

(21) Appl. No.: 14/312,967

(22) Filed: Jun. 24, 2014

Related U.S. Application Data
(60) Provisional application No. 61/838,344, filed on Jun. 24, 2013.

(51) Int. Cl. A61N 7/00; A61M 37/00

(52) U.S. Cl. CPC A61N 7/00; A61M 37/0092; A61N 2007/0043; A61N 2007/0056; A61M 2205/0216
USPC 601/2

(57) ABSTRACT

Embodiments isolate exposed surfaces to increase the effectiveness of different treatment modalities. Embodiments isolate hollow spaces within the body to increase the effectiveness of ultrasound energy and/or other treatments. Such hollow spaces within the body may include nasal surfaces, and recessed or sequestered surfaces, e.g. sinus cavity surfaces or other anatomical structures, such as upper and lower gastrointestinal tract, airways, uterine and vaginal cavities and the anorectal canal, for example. Isolating the area to be treated reduces the volume of the enclosed and delimited space and reduces the tissue surface against which the ultrasound and/or biologically active substances act. For example, isolating a hollow passageway within the body enhances the effectiveness of ultrasound within the isolated space, and constrains the biologically active fluid and/or the gel or fluid configured to conducts the ultrasonic energy from the emitter thereof (e.g., an ultrasound waveguide) to the surfaces to be treated.

This application claims benefit of U.S. Provisional Patent Application Ser. No. 61/838,344 entitled “METHODS, DEVICES AND SYSTEMS FOR INCREASING THE EFFECTIVENESS OF ULTRASOUND AND OTHER TISSUE TREATMENT MODALITIES” filed on Jun. 24, 2013, the disclosure of which is incorporated by reference herein in its entirety.

### BACKGROUND

At the interface of the external environment, thick with infectious organisms, and the body's barrier surfaces, skin and wet mucous membrane, infections can occur, fester, invade and spread. The body has multiple innate and acquired mechanisms to isolate, control and usually destroy infectious invasion. Today we depend on antibiotics and other chemicals to be administered orally or by vein to dilute through the blood stream as it is carried throughout. A portion of the medication arrives at the infection site and must diffuse from the vasculature to the surface where the infection is active, with a limit to the concentration possible that may not be effective. At the same time, these antibiotics spread to other non-infected surfaces where the effects are not trivial, such as colitis associated with systemic antibiotic use. The result may end up as not enough treatment at the infected site, time for bacteria to develop resistance and secondary effects where there is no disease (e.g. colitis).

In some instances, topical antibiotics have been advocated. Three major problems are seen with this approach. One, on an open flat surface, the topical medication cannot be contained at the site of infection. Two, in a closed cavity, a large amount of antibiotic fluid must be used. Three, in either case, there is no ability to move the fluid to either drive the medication closer to the infection or administer fresh fluid.

Bacterial sinusitis, for example, is an extremely common event that is associated with viral rhinitis and is treated with antibiotics. At least 37 million people in the United States are infected every year and of those, at least 2 million are not cured. As the use of antibiotics has spread, bacterial resistance has grown to a public health scourge.

Bacteria are known to have two different phenotypes and can switch between the two. One, called planktonic, involves bacteria living as single organisms. They are in a state of high metabolic activity as they live and interact with their environment. It is in this form that bacteria are the most susceptible to attack by antibiotics, chemicals of various types, changes in oxidative moieties within and energy assaults from without. The second phenotype is called biofilm. The individual bacteria are in a low metabolic state, are isolated from the environment by a slimy, polysaccharide film (EPS), receive nutrients and chemical signals and DNA information from other bacteria through tunnels in the EPS. This biofilm is attached to a surface. Biofilm has a “life cycle” in that EPS over a period of 9-12 days as it matures to produce planktonic daughter cells that burst from the biofilm and disperse to set up new biofilm colonies. This phenotype has been proven to be very resistant to treatment. Finally, bacteria are also known to enter host surface cells by phagocytosis without being destroyed and live within the cell in a parasitic fashion. When the epithelial cell dies, it releases these bacteria as a planktonic organism to invade other cells or set up new biofilms. Thus, biofilms and parasitized epithelial cells become reservoirs of planktonic cells spreading the infection. Bacteria in the planktonic phase are most susceptible to antibiotics. They and their related chronic forms (biofilm and parasitic) should be approachable targets for topical therapy.

### BRIEF DESCRIPTION OF THE DRAWINGS

FIG. 1A shows a hollow body cavity whose internal volume has been decreased for administration of ultrasonic energy or another treatment modality, according to one embodiment.

FIG. 1B shows a hollow body cavity whose internal volume has been decreased for administration of ultrasonic energy or another treatment modality, according to one embodiment.

FIG. 2 is a diagram of aspects of one embodiment.

FIG. 3A is a diagram of further aspect of one embodiment.

FIG. 3B is a diagram showing volume reduction within an already bounded cavity or biological conduit, according to yet another embodiment.

FIG. 4A is a diagram illustrating use of an isolation system and method using an endoscopic system, according to one embodiment.

FIG. 4B is a diagram illustrating use of an isolation system and method using an endoscopic system, according to one embodiment.

FIG. 4C is a diagram illustrating use of an isolation system and method using an endoscopic system, according to one embodiment.

FIG. 4D is a diagram illustrating use of an isolation system and method using an endoscopic system, according to one embodiment

FIG. 5A shows one embodiment, as applied to the treatment of a nasopharyngeal cavity.

FIG. 5B shows one embodiment, as applied to the treatment of a nasopharyngeal cavity.

FIG. 6 shows further aspects of one embodiment, as applied to the treatment of a nasopharyngeal cavity.

FIG. 7A shows cavity isolation for the treatment of sinusitis, according to one embodiment.

FIG. 7B shows cavity isolation for the treatment of sinusitis, according to one embodiment.

FIG. 8A shows further aspects of cavity isolation for the treatment of sinusitis, according to one embodiment.

FIG. 8B shows further aspects of cavity isolation for the treatment of sinusitis, according to one embodiment.

FIG. 9A is a diagram illustrating isolation and target area volume reduction for intrauterine surface treatment, according to one embodiment.

FIG. 9B is a diagram that illustrates isolation and target area volume reduction for intrauterine surface treatment, according to one embodiment.

FIG. 10A is a diagram showing isolation and target volume reduction for the treatment of jeopardized surfaces of the upper gastro-intestinal tract, according to one embodiment.

FIG. 10B is a diagram showing isolation and target volume reduction for the treatment of jeopardized surfaces of the upper gastro-intestinal tract, according to one embodiment.

FIG. 11 is a diagram showing isolation and target volume reduction for the treatment of jeopardized surfaces of the lower gastro-intestinal tract, according to one embodiment.

FIG. 12A is a diagram showing isolation and target volume reduction for the treatment of jeopardized surfaces of the upper airway, according to one embodiment.

FIG. 12B shows isolation and target volume reduction for the treatment of jeopardized surfaces of the upper airway, according to one embodiment.

FIG. 13 shows isolation and target volume reduction for the treatment of jeopardized surfaces of the anorectal canal, according to one embodiment.

FIG. 14 shows a device according to one embodiment, deployed in an exemplary conduit or cavity within biological tissue.

FIG. 15 shows a device according to a further embodiment, deployed in an exemplary cavity within biological tissue.

### DETAILED DESCRIPTION

Embodiments, in one aspect, isolate exposed (e.g., jeopardized or diseased) surfaces to increase the effectiveness of different treatment modalities. For example, embodiments may be configured to isolate hollow spaces within the body to increase the effectiveness of for example, ultrasound energy or other treatments. Such hollow spaces within the body may include, for example, nasal surfaces, and recessed or sequestered surfaces, e.g. the sinus cavity surfaces that extend from the nasal passages. Embodiments are applicable to a plurality of other anatomical structures, such as upper and lower gastrointestinal (GI) tract, airways, uterine and vaginal cavities and the anorectal canal, to identify but a few of the possible sites. According to embodiments, isolating the area to be treated reduces the volume of the thus-enclosed and delimited space and reduces the tissue surface against which the ultrasound and/or biologically active substances act. For example, isolating a hollow passageway within the body enhances the effectiveness of ultrasound within the isolated space, and constrains the biologically active fluid and/or gel or fluid configured to conducts the ultrasonic energy from the emitter thereof (e.g. an ultrasound waveguide) to the surfaces to be treated. Within the present context, the fluid may, according to embodiments, have a predetermined viscosity and may resemble a gel or a free-flowing fluid.

Embodiments may be applied to isolate at least three different types of structures within the body such as, for example 1) spaces comprising air or fluid ingress and egress openings such as the nasal passage; 2) within a cavity in which the volume is large relative to the inner surface to be treated and in which only the inner surface of the space requires treatment (e.g. a nasal sinus); and 3) an exposed, more or less flat surface (e.g., cheek mucosa) where a limited area tissue is infected, necrotic or otherwise in need of treatment. Although the following description is presented in terms of ultrasound treatment of tissues, it is to be understood that such is exemplary implementation is for illustrative purposes only and that embodiments are not to be limited to ultrasound. For example, radio-frequency (RF) energy may be used to good effect, as may cryo-ablative techniques, laser, plasma, photo- and sono-dynamic therapies and/or any other treatment modality that may benefit from delimiting, constraining or otherwise reducing the volume of treatment and/or the surface area of tissue to be treated. For example, any bodily structure in which a localized area is affected and in need of treatment and in which isolation is possible is a good candidate for application of the present embodiments.

According to embodiments, by isolating and/or decreasing the area or volume to be treated before ultrasound energy is applied to the affected area, the ultrasonic energy level may be maintained at sufficient levels to kill bacteria in whatever form. This energy may be transmitted from the ultrasound waveguide to the affected tissues through any suitable medium, such as an acoustically-conductive mist, gel or other fluid. For a space such as the nasal cavity, the space may be limited by a variety of different volume-limiting structures such as, for example, one or more balloons, swellable sponges, expandable stents and the like. When a volume is bounded in this manner, ultrasound energy and/or fluids or mist used are prevented from escaping and reducing the effectiveness of the treatment. A flat exposed surface may also be bounded by such volume-limiting structures for increased effectiveness of whatever treatment modality is used. In the case in which a bodily cavity is to be treated, the central portion of the space may be cleaned (e.g. of scar tissue and/or pooled pus) to expose and limit treatment to the infected or affected surface lining that actually requires treatment. According to one embodiment, the space over the surface lining of the affected tissue may be limited to a thin rim or annulus, layer or volume, so as not to allow undue dissipation of the delivered energy (e.g., ultrasonic, RF or thermal). According to one embodiment, one or more judiciously-placed balloons, sponges or stents within the space concentrates the applied energy or any energized mist on the cavity's surface. This intensifies and maintains the beneficial activity (e.g. cavitation, micro-streaming, etc.) of the applied ultrasound. For example, to best transmit the applied energy to the tissue surface to be treated, a conducting fluid, a gel or an energized mist may be introduced into the bounded and isolated cavity or space. Such fluids, gel, mists or atomized fluid may, for example, comprise simple saline, which may be doped with antibiotics or one or a plurality of chemicals or biologically active substance such as antimicrobials or steroids that, once activated, effectively kill bacteria and fingi.

Indeed, because region or volume is isolated according to embodiment, liquids and gels configured for specific purposes may be utilized to good effect. Indeed, fluids may be configured and chosen according to the treatment envisaged. For example, the fluid may have antibacterial properties to kill specific bacteria, anti-inflammatory properties to halt the inflammatory reaction to infection, or may be carrier or vector for the administration of specific (and often costly) biologicals configured to cause sophisticated targeted treatments and tissue manipulation. According to embodiments, the liquid or gel may be configured with active ingredients configured for anti-cancer treatments with chemotherapeutics, anti-vascular growth factors, biological and radioactive localized brachiotherapy as well as radiotherapy. In addition, the isolation and treatment opportunities afforded by embodiments enable localized treatment via cryotherapy and thermal therapies.

The gels and other transmission substances can spread the ultrasound or other energy modalities into all the nooks and crannies of really any surface. This can be both if placed inside of a balloon and also if placed outside of the balloon in the thin space overlying the tissue surface. This latter would also fit with the devices that enclose a tubular structure such as a bowel or in case of the nose all of the nasal cavity.

Accordingly, one embodiment of a method of treating tissue may comprise isolating a bodily space, volume or structure, filling the isolated space, volume or structure with a fluid that conducts ultrasonic energy and/or that activates any chemicals (e.g., one or more biologically active materials) in the fluid, and applying the ultrasonic energy in the thus-delimited space, with consequent concentration of the applied energy and chemicals to the isolated area. Other embodiments may be implemented in which other forms of energy are applied to the isolated space, volume or structure.

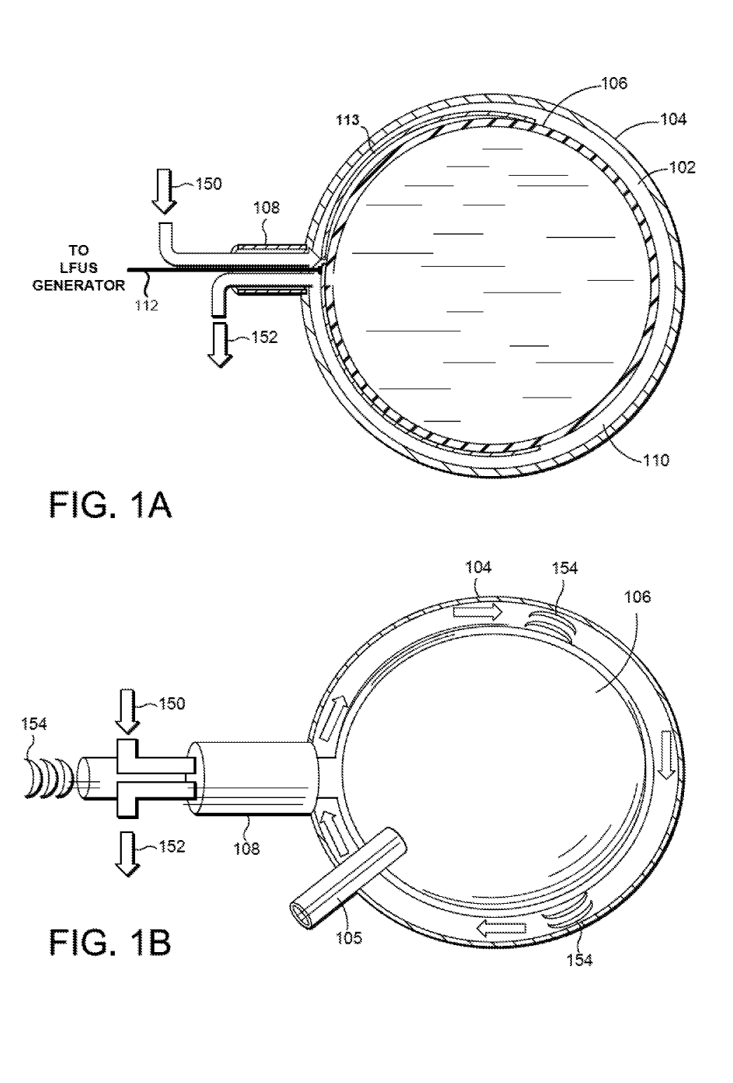

Turning now to the figures, FIG. 1A shows a hollow body cavity whose internal volume has been decreased for administration of ultrasonic energy or another treatment modality, according to one embodiment. As shown therein, a hollow space 102 within the body may be defined by an anatomical wall 104 consisting of an epithelial layer of cells and a submucosal layer of mesenchymal tissue. As shown, a catheter or trocar 108 may be inserted within the hollow space 102 and a balloon 106 inserted therein and inflated within the hollow space 102. According to one embodiment, the balloon 106 may comprise Mylar®, plastic, elastomeric or polymeric material and may be inflated such that the outer surface thereof is in intimate contact with the anatomic wall 104. In practice, bodily fluids may naturally fill the thin interstitial space 110 between the anatomic wall 104 and the balloon 106. It may also be advantageous to add an antibiotic or antimicrobial or other solution or mixture to this area to enhance the effects of the ultrasound, as detailed above. The resulting aqueous environment in this interstitial space may be effective in transmitting ultrasonic energy generated by ultrasonic wire 112 into the interstitial space 110 without unacceptable levels of dissipation thereof. Wire 112 may be coupled to an ultrasound coupling 113 at least partially surrounding the balloon 106 or otherwise disposed within the interstitial hollow space 102. This ultrasound coupling 113 may comprise a wire or other ultrasonically-conducive element or structure. For example, the ultrasound coupling 113 may be configured as a net or mesh-work that partially or substantially fully encircles the balloon 106 and that conducts ultrasound energy applied thereto by wire 112. Ultrasound coupling 113 may be localized within hollow space 102 and with respect to balloon 106 to focus LFUS on a particular region of the enclosed and isolated volume. If fully encapsulating the balloon 106, the conducted ultrasonic energy may be radiated within substantially omni-directionally, to treat all tissues within the bounded volume.

The bodily fluids occupying the interstitial space 110 may be suitable for supporting the beneficial mechanical action of the micro-bubbles created by cavitation, acoustic micro-streaming, induced vasodilation, perfusion, hyperthermia via cavitation and cycling pressures, induced cell membrane permeation and/or the production of nitrous oxide (NO) and other reactive species (e.g., e.g. oxygen singlets, hydroxyl ions, etc. produced by the energy source) and/or other effects caused by the application of the ultrasonic energy. To generate such micro-bubbles and cavitation, a low frequency ultrasound (hereafter, “LFUS”) waveguide may be inserted within the catheter or trocar 108, with the ultrasound generating tip or waveguide disposed within, according to one embodiment, the interstitial space 110 between the balloon 106 and the anatomic wall 104.

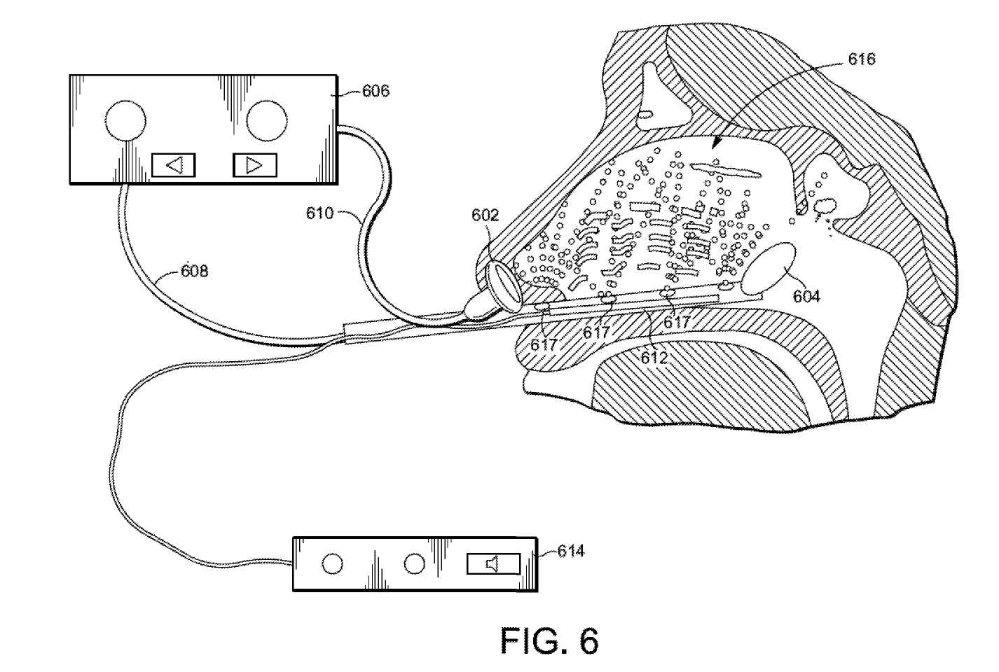

For example, the LFUS may be generated from an ultrasound generator using piezoelectric transducers to which signals are applied. The frequency, power, amplitude, waveform and/or on/off duty cycle of the signals applied to the ultrasound emitter of the LFUS generator may be selected as desired. Generally, the LFUS may be generated with sufficient voltage and power to create ultrasound having sufficient energy to treat the target tissue. For example, the signals applied to the ultrasound transducer may be such as to cause the ultrasound transducers to generate ultrasonic energy within a frequency range of about 20 kHz to about 40 kHz. According to one embodiment, the low frequency ultrasound generator (such as shown at 614 in FIG. 6) may be configured, for example, to output a peak-to-peak voltage from about 40V to about 160V. For example, the generator may be configured to output a peak-to-peak voltage from about 20V to about 160V and a duty cycle between about 10% and 100%. For example, the generator may be configured with an output power of up to about 25 watts with a peak-to-peak output voltage of about 80V, when tuned at resonance. According to one embodiment, the piezoelectric (magneto or other type of) transducers of a suitable LFUS generator may be configured to generate ultrasonic energy between 23 kHz and 28 kHz. The LFUS generator, according to one embodiment may be provided with inertial blocking functionality and a protective concentrator. For example, the transducers of the LFUS generator may be configured to generate ultrasonic energy at about 25 kHz. Within the present context, the qualifier “about” may be interpreted as + or −20%.

The methods, devices and systems disclosed herein may also be used to good advantage with higher frequencies. For example, the methods, devices and systems disclosed herein may be use higher frequencies, such as in the MHz range. Such MHz frequencies, for example, may be used for intra-arterial work. The isolating and volume-reducing methods, devices and systems may, therefore, be applied to procedures at other frequencies, such as the higher MHz-range frequencies.

According to one embodiment, in addition to any bodily fluids that may be present between the balloon 106 and the anatomic wall 104, such interstitial space may be filled with an ultrasonic conductive fluid, that is, a fluid or gel that is a good conductor of ultrasonic energy and that promotes the generation of acoustic micro-streaming, cavitation, micro-bubbles, induced vasodilation, perfusion, hyperthermia via cavitation and cycling pressures, induced cell membrane permeation and/or the production of NO and other reactive species and/or other effects. According to a further embodiment, the balloon may be inflated with air or an inert gas such as carbon dioxide. According to another embodiment, the balloon 106 may be inflated with a conductive fluid or gel, as may be the interstitial space 110. In this case, the distal, ultrasound-transmitting LFUS waveguide or wire may be inserted within the interstitial space 110 or indeed directly within the balloon 106. In this case, the ultrasonic energy may be transmitted within the ultrasonically conducting fluid, gel or other material filling the balloon 106, across the membrane thereof to the anatomic wall 104 in intimate contact therewith, either directly or through a thin interstitial space between the anatomic wall 104 and the outer membrane of the balloon 106. In this manner, substantially the entire outer surface of the balloon 106 becomes an ultrasonic generator, radiating to the facing targeted biological surfaces. According to one embodiment, to maintain the desired efficacy, the frequency, power, amplitude, waveform and/or on/off duty cycle of the signals applied to the ultrasound emitter of the LFUS generator may be suitably modified to account for reflections off the targeted biologicals surface and the balloon 106, among other effects.

According to one embodiment, the balloon 106 (however inflated) may serve to reduce the effective volume through which the LFUS energy need be transmitted to thereby increase its effectiveness without resorting to higher but sub-optimal power levels to achieve the desired beneficial action on the affected tissue. This reduced volume also serves to concentrate any biologically-active fluids delivered to the interstitial space 110. Indeed, in the absence of such volume-reducing balloon 106, the concentration of such biologically-active fluid would necessarily decrease as it dispersed within the larger volume, with necessarily decreased beneficial action.

FIG. 1B shows a hollow body cavity whose internal volume has been decreased for administration of ultrasonic energy or another treatment modality, according to one embodiment. As shown, the volume within the cavity defined by the cavity surface 104 may be decreased by inflating a balloon 106 therein. The balloon 106 may be inflated through a balloon inflation port 105. The port 105 may be provided within or coupled to the present device. Acoustically-transmissive fluid, whether doped with a therapeutically-beneficial agent or not, may be delivered to the interstitial space between the inflated balloon 106 and the cavity surface 104 at 150 (fluid inflow), and evacuated at 152 (fluid outflow). The fluid so delivered may be caused to circulate within the aforementioned interstitial space, as illustrated by the arrows 150, 152 and the arrows between the cavity surface 104 and the outer surface of the inflated balloon 106. The (e.g., LFUS) energy delivered through the catheter or trocar 108 may, therefore, propagate within the entire interstitial space between the balloon 106 and the cavity surface 104. Other placements of the inflated balloon or other volume-isolating structure may serve to delimit specific surfaces of the cavity. If the source of ultrasound were to be placed within the balloon 106, the ultrasonic energy may propagate through the fluid or gel within the balloon 106 to the surface 104 of the cavity formed within the bone, cartilage or soft tissue, as shown at 154. For example, if the balloon were to be pushed against one region of the cavity surface, only those surfaces thereof not in contact with the inflated balloon would receive appreciable LFUS energy. As shown, the balloon or other volume or surface delimiting structure is effective to reduce the treatment space, to thereby enable effective treatment with, for example, ultrasound and treatment fluid, gel or material. By reducing the treatment volume, the concentration of the LFUS is increased, thereby increasing the effectiveness of the treatment. In practice, the balloon 106 may be inserted into a natural or created cavity in at least a partially deflated state, and fluid or gel, for example, may be introduced into the balloon and/or the interstitial space between the balloon 106 and the cavity surface 106. LFUS may then be applied to the interstitial space and/or to the interior of the balloon 106 to treat the cavity surface 104. Thereafter, the LFUS may be turned off, the waveguide removed, the balloon deflated and the interstitial space evacuated through fluid outflow port 152.

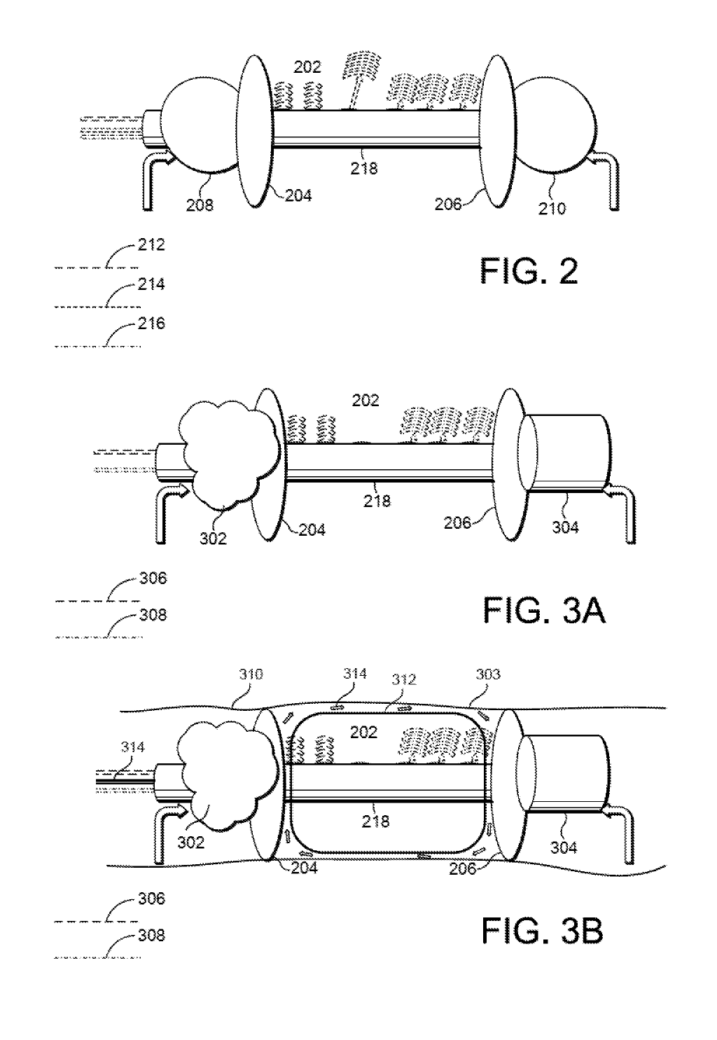

FIG. 2 is a diagram of aspects of one embodiment. Shown therein is a depiction of a catheter or trocar 218 inserted into a hollow space 202 within a body such as, for example, a nasal cavity. As is known, the nasal cavity is not a naturally bounded structure. Indeed, the volume thereof defines openings such as the external naris and the opening of the nasopharynx. Therefore, introduction of ultrasonically-conductive fluid into the nasal cavity would be ineffective, as the fluid would tend to run out of the nose at the proximal end and through the nasopharynx at the distal end of the cavity. Such physiological openings are stylistically shown in FIG. 2 at 204 and 206. According to one embodiment, the nasal cavity may be isolated and bounded by volume-isolating structures 208, 210. Such volume-isolating structures may comprise, for example, any combinations of balloon, stents, or sponges or other swellable, expandable or otherwise deployable opening obstructing or limiting structures. For example, volume-isolating structure 208 may comprise a balloon or other inflatable device and the volume-isolating structure 210 may comprise a swellable sponge or a stent. Such volume-isolating structures 208, 210 may be separate from the LFUS delivery device and may be placed before the procedure, and the resultant bounded volume filled with ultrasonically-conductive fluid and/or other biologically active substances and LFUS applied within the so-bounded volume, as suggested at 212, 214, 216.

According to one embodiment, a catheter or trocar 218 may be inserted into the cavity 202. The catheter or trocar 218 may comprise or be coupled to, according to one embodiment, both the proximal volume-isolating structure 208 and the distal volume-isolating structure 106 coupled thereto. Once the catheter or trocar 218 is in place within the volume 202, the volume-isolating structures 208, 210 may be expanded, by filling them with ultrasonically-conductive fluid, by causing them to naturally expand within the aqueous environment within volume 202 or through introduction of ultrasonically-conductive fluid and/or other biologically-active fluids, gels and the like. In the case in which one or more of the volume-isolating structures 208, 210 comprise a mechanical component such as stents, such may be actuated after insertion thereof into the cavity, to effectively close off and bound the volume 202. This bounds the volume of interest (e.g. the volume in which the affected tissues are located), to thereby enable the optimal transmissivity of the ultrasonic energy to the affected tissues. Both the interstitial space between the tissue wall and the catheter or trocar 218 and the interior lumen of the catheter or trocar 218 may be filled with ultrasonically-conductive fluid or gel.

An LFUS-generating structure such as an encapsulated piezoelectric (PZ) device, LFUS waveguide or wire may then be activated. The LFUS-generating structure may be disposed within the internal lumen of the catheter or trocar 218, with the generated LFUS energy escaping from suitably sized and located openings or perforations 220 therein, as suggested at 216. Alternatively, the LFUS-generating structure may be disposed against or within the one of the volume-isolating structures, with the generated acoustic energy of the LFUS being transmitted through the volume-isolating structure, directly into the bounded volume 202 or into the interior lumen of the catheter or trocar 218 and escaping through one or more openings or perforations 220 defined within the wall of the catheter or trocar 218. Alternatively still the LFUS-generating structure (e.g., waveguide) may be inserted within the catheter or trocar 218 and steered to emerge therefrom through one or more perforations or openings into the interstitial space between the catheter or trocar 218 and the tissue surface of the bounded volume, to directly deliver the acoustic energy to the affected tissue (through any bodily fluids or acoustically-transmissive fluid that may be present therein), as suggested at 214 in FIG. 2.

FIG. 3A is a diagram of further aspects of one embodiment. As shown therein, the volume-isolating structures 302, 304 need not be identical. Indeed, each of the volume-isolating structures 302, 304 may be configured and/or conform to the opening to which they are to be applied. For example, a proximal volume-isolating structure may comprise a swellable sponge that is well adapted to proximal structures of the volume to be bounded, whereas the distal volume-isolating structure 304 may comprise, for example, a balloon or a stent, which may be better suited to close a distally-disposed opening. As shown at 306, the LFUS waveguide may be placed within the internal lumen of the catheter or trocar 218, and the LFUS acoustic energy generated thereby may propagate across the wall of the catheter or trocar 218. Alternatively, as shown at 308, the LFUS generator may be directly coupled (e.g., through a mechanical coupling) to the material of the catheter or trocar 218 (e.g., a polymer) or even to one of the volume-isolating structures 302, 304, if such are acoustically transmissive. The polymer of the conduit, then, may transmit the energy (e.g., acoustic, LFUS) into the cavity.

FIG. 3B is a diagram showing volume reduction within an already bounded cavity or biological conduit, according to yet another embodiment. For example, reference 303 may correspond to the walls of a biological conduit such as, for example, the walls of the GI tract, among many other possibilities. The volume isolating structures 302, 304 produce a closed cavity within the biological conduit. The volume within the biological conduit 303 bounded by volume-isolating structures 302, 304 may be further decreased, according to one embodiment, by a further expandable member 312 disposed on or integral to catheter 218. According to one embodiment, this further expandable member 312 may comprise an inflatable balloon that may be configured to expand to or just short of the walls of the passageway 313 that has been closed or blocked off. The balloon 312 may be configured to adhere to the walls of the passageway 313 or may be configured to leave an interstitial space between the balloon 312 and the walls of the passageway 312. The balloon 312 may be filed with an acoustically-transmissive fluid or gel via delivery lumen 314. Similarly, the interstitial space may be filled with gel configured to conduct low frequency ultrasound to the wall. IN this manner, LFUS may be generated through catheter 218, travel through the LFUS-conducting fluid or gel within balloon 312, through the LFUS-conductive fluid or gel in the interstitial space between the outer surface of the balloon 312 and the facing surface, to the target tissues. As suggested at 314, fluids may be caused to circulate over the balloon, between the outer surface of the balloon and the walls of the bounded cavity through delivery and evacuation ports. According to one embodiment, the balloon 312 may be configured to expand to intimately conform to the interior of the tubular cavity closed by the volume-isolating structures 302, 304 and conduct LFUS. Such a system may be advantageously applied to the nasal passage also.

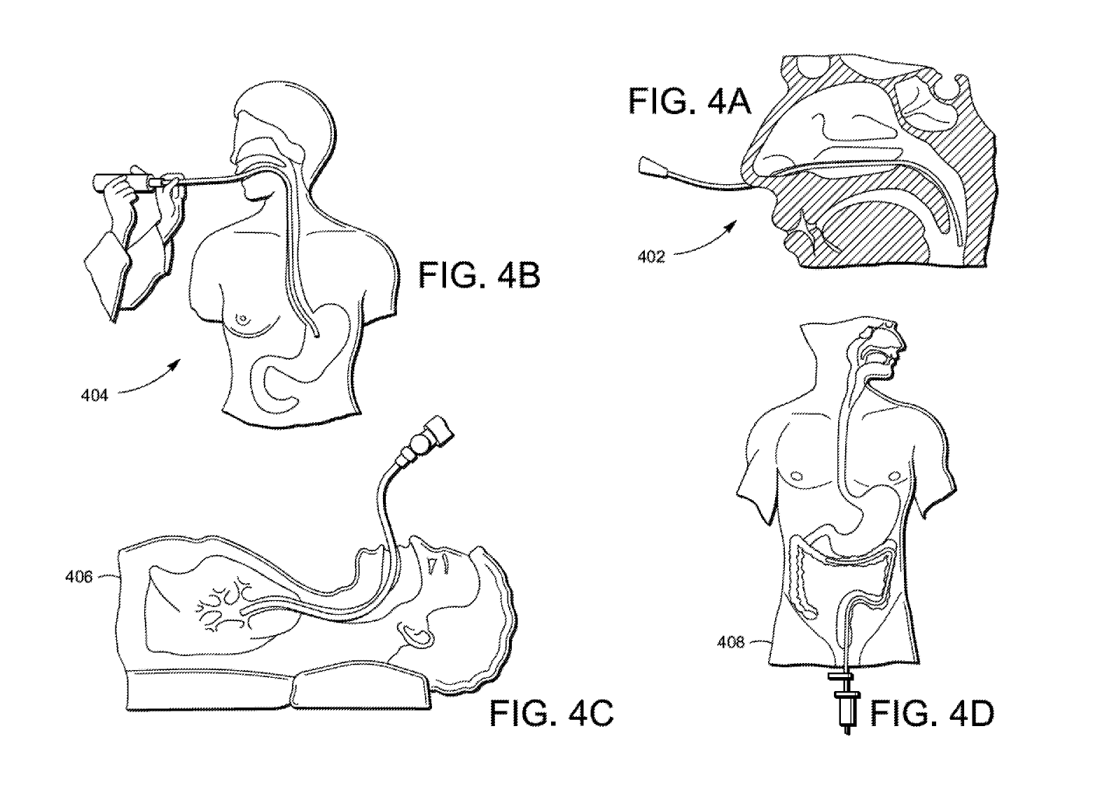

FIGS. 4A-4D are diagrams illustrating use of embodiments of the present isolation system and method in combination with an endoscopic system, according to one embodiment. Such endoscopic applications may include, for example, nasal endoscopy 402 as shown in FIG. 4A, gastroscopy 404 as shown in FIG. 4B, bronchoscopy 406 as shown in FIG. 4C or colonoscopy 408 as shown in FIG. 4D.

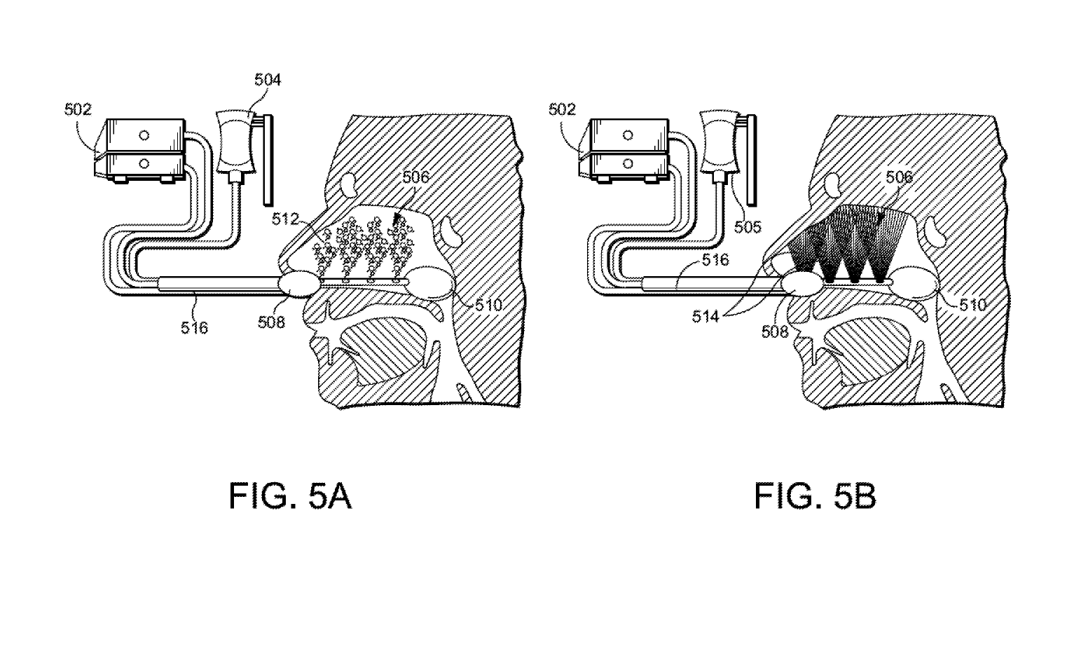

FIGS. 5A and 5B show one embodiment, as applied to the treatment of a nasopharyngeal cavity. As shown therein, a catheter or trocar 516 may be inserted into the nasopharyngeal cavity 506. A distal volume isolating structure 510, such as a balloon, sponge stent or plug, may block (that is, at least partially obstruct) the nasopharynx and a proximal volume-isolating structure 508 may be disposed so as to block (plug, at least partially obstruct) the nasal passages. The nasal passage and nasopharyngeal cavity 506 is now bounded and isolated. In this configuration, the nasopharyngeal cavity 506 may support a volume of ultrasonically-transmissive fluid 504 without the fluid running out of the patient's nose or larynx. As shown, an LFUS generator 502 may now be activated, to generate LFUS in the bounded nasopharyngeal cavity 506. As shown in FIG. 5A, micro-bubbles 512 may then be generated though cavitation of the ultrasonically-transmissive fluid 504. Such micro-bubbles and other physical manifestations of the applied LFUS have a scrubbing action, and may be effective to scrub the (e.g. mucosal) surfaces of bacteria or biofilm. Detritus loosened by the simultaneous delivery and evacuation of fluid from the cavity under treatment may be evacuated, as suggested in FIG. 1B. Moreover, the micro-bubbles generated by the LFUS-induced cavitation and emitted from openings in the catheter or trocar 516 may cause the mucosa, the surfaces of the nasopharyngeal cavity 506, to become cleaned, raw and highly vascularized. In such a state, the surfaces of the nasopharyngeal cavity 506 are primed to receive further treatment, such as shown at 514 in FIG. 5B, in the form of, for example, a mist of a biologically active substance, such as an antibiotic, an antimicrobial agent or steroids, for example. Other materials may be delivered, such as dyes, sono-sensitizers, photo-sensitizers, antiseptic preparations or other therapeutically beneficial fluids. According to embodiments, the liquid or gel may be configured with active ingredients configured for anti-cancer treatments with chemotherapeutics, anti-vascular growth factors, biological and radioactive localized brachiotherapy as well as radiotherapy. In addition, the isolation and treatment opportunities afforded by embodiments enable localized treatment via cryotherapy and thermal therapies. Such fluids may become activated through the subsequent application of RF, ultrasound, laser light or other forms of energy. After the procedure, the proximal and distal cavity-isolating structures 508, 510 may be removed, after optionally deflating them or compressing them by pulling at least a portion thereof back into the lumen of the catheter or trocar 516.

FIG. 6 shows further aspects of one embodiment, again as applied to the treatment of the nasopharyngeal cavity. As shown, a fluid management complex 606 may be provided to supply, as shown at 608 and evacuate, as shown at 610, fluids from the hollow body cavity or other biological conduit under treatment. As shown, a catheter or trocar 612 may be inserted into the nasopharyngeal cavity 616. According to one embodiment, volume-isolating structures 602, 604 may be coupled to the catheter or trocar 612, to isolate the nasopharyngeal cavity 616 from other anatomical structures and to reduce and bound the volume in which subsequent treatments are carried out. The distal (posterior) volume-isolating structure 604 may comprise, for example, a swellable sponge, a plug, an inflatable balloon or a mechanical device, such as a stent. For example, a volume-isolating plug may be formed of a silicone medical grade elastomer such as, for example. Silastic® MDX4-4210. Silicone is not overly heat sensitive, is not destroyed by vibrations and exhibits high elasticity.

The proximal (anterior) volume-isolating structure 602 may also comprise one or more balloons, swellable sponge or mechanical device. Moreover, as shown in the embodiment of FIG. 6, the proximal volume isolating structure 602 may be generally dome or funnel-shaped and configured with an internal lumen enabling it to also deliver or evacuate fluids from the nasopharyngeal cavity 616 or other volume under treatment. A LFUS generator 614 may be coupled to the catheter or trocar 612, for the delivery of acoustic energy through a suitable waveguide. The catheter or trocar 612 may define openings 617 through which at least the LFUS energy may be delivered to the nasopharyngeal cavity 616.

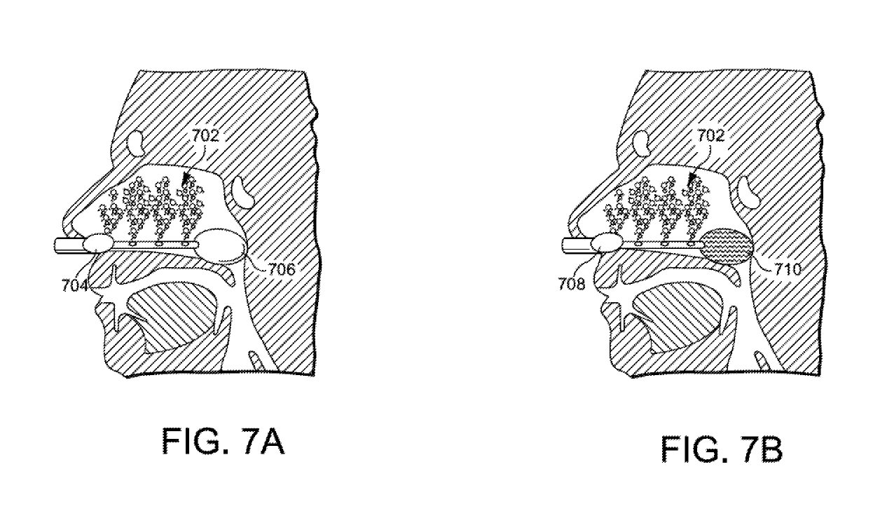

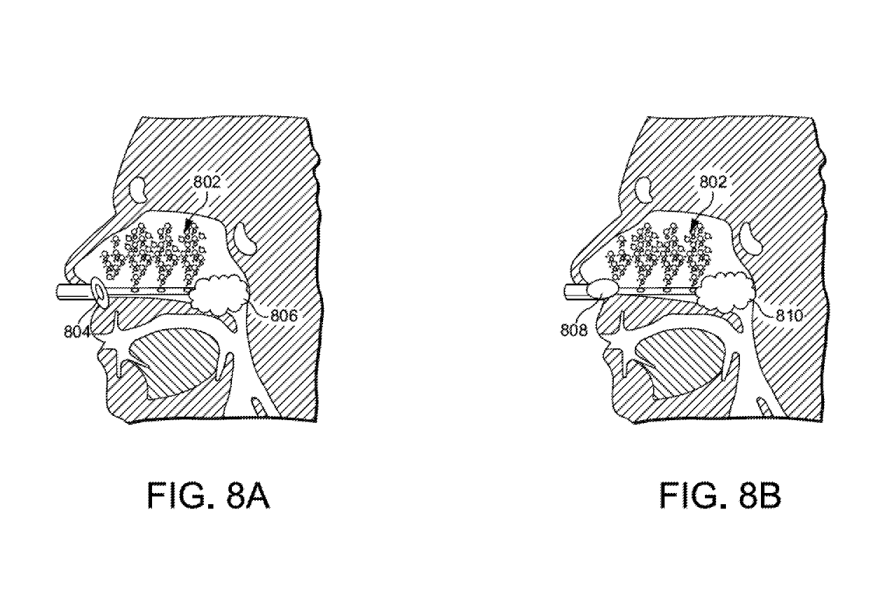

FIGS. 7A and 7B show cavity isolation for the treatment of sinusitis, according to one embodiment. As shown in FIG. 7A, the nasal and nasopharyngeal cavity 702 may be isolated using two similar volume-isolating structures, such as two inflatable balloons 704, 706 or two or more dissimilar volume-isolating structures, such as inflatable balloon 708 and an expendable stent 710 as shown in FIG. 7B. Similarly, FIGS. 8A and 8B show further aspects of cavity isolation for the treatment of sinusitis, according to one embodiment. As shown, the nasopharyngeal cavity 802 may be isolated using a volume-isolating structure 804 configured for suction and/or irrigation and/or other circulation processes. For example, the volume-isolating structure 804 may have a ring, dome or funnel shape that may assist in fluid suction, irrigation or other circulation processes. The volume-isolating structure 804 may also be inflatable. The distal volume-isolating structures 806, 810 in FIGS. 5A and 8B may comprise, for example, one or more polymeric sponges exhibiting a high swelling ratio in an aqueous environment. Other combinations are possible, according to embodiments.

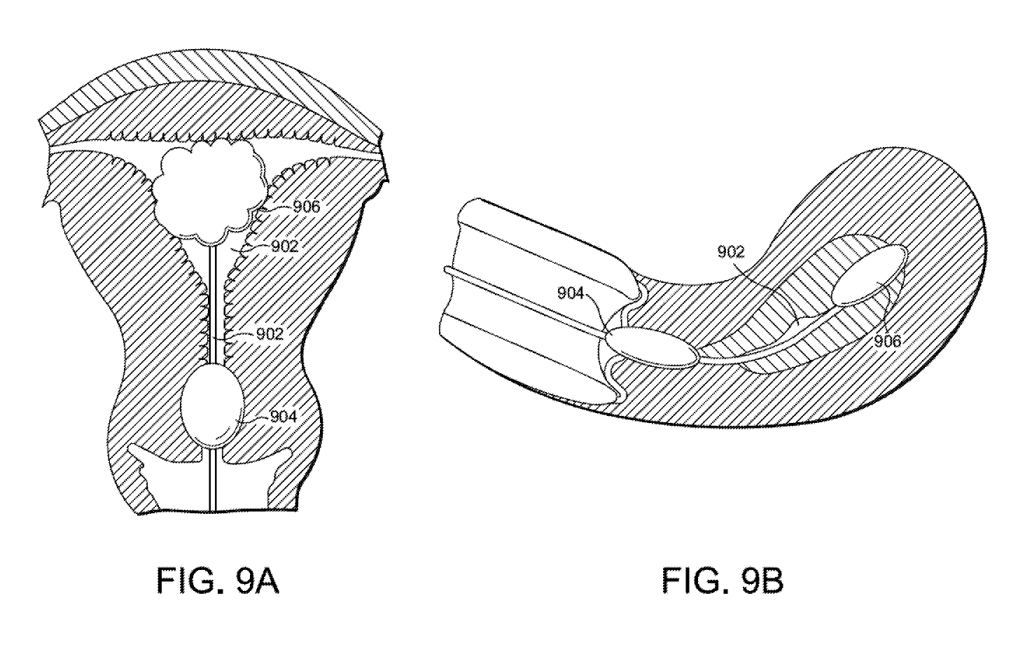

FIGS. 9A and 9B show different view of an embodiment configured for the treatment of intrauterine surfaces. As shown in FIGS. 9A and 9B, the intrauterine volume to be isolated is shown at 902. As shown, the catheter or trocar 902 comprising the LFUS waveguide may be inserted through the vaginal opening into the vaginal canal, past the cervix and into the uterus. The catheter or trocar 902 may be flexible, for ease of insertion and patient comfort. The catheter or trocar 902 may also be configured to be somewhat steerable. According to one embodiment, the distal volume-isolating structure 906, such as a swellable polymeric sponge, may be advanced distally into the uterus, while the proximal volume-isolating structure 904, such as an inflatable balloon, may be disposed at or near the cervix, thereby confining the uterine surface to be treated between the distal and proximal volume-isolating structures 904, 906. The catheter/trocar 902 may be configured to be locally expandable or swellable in the aqueous environment in which it is placed, so as to enable a selective isolation of even smaller portions of the uterine surface.

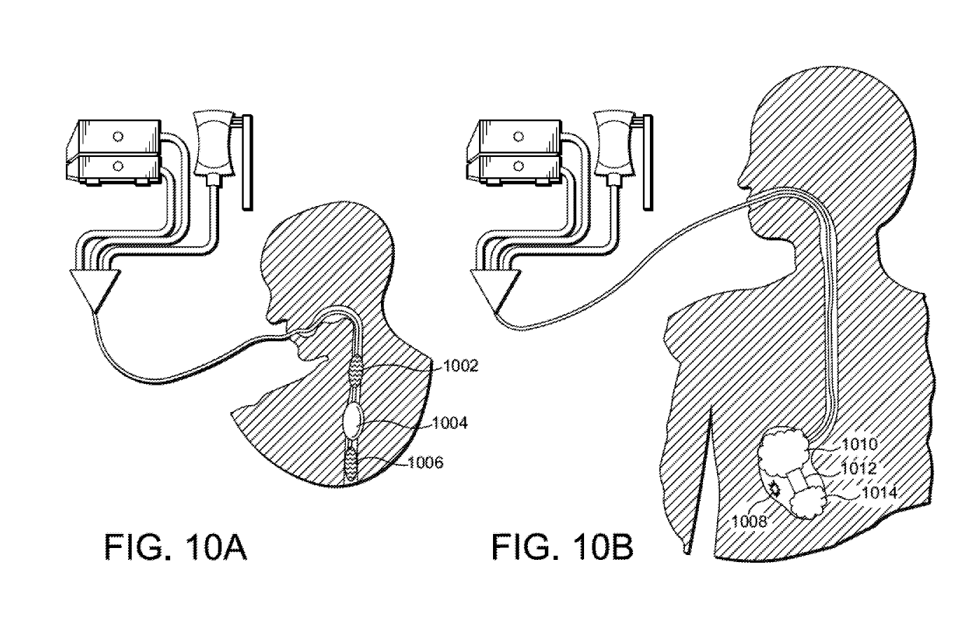

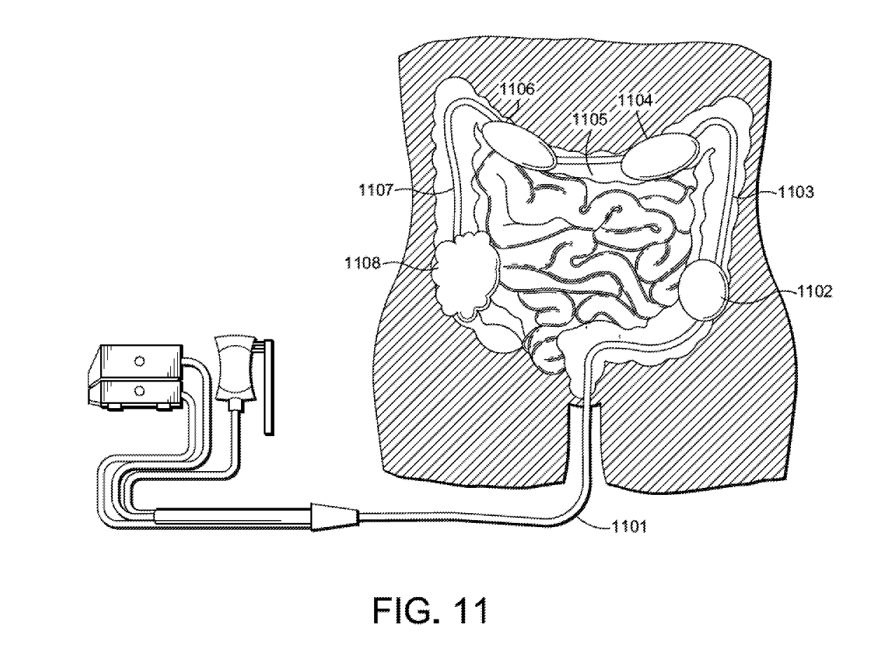

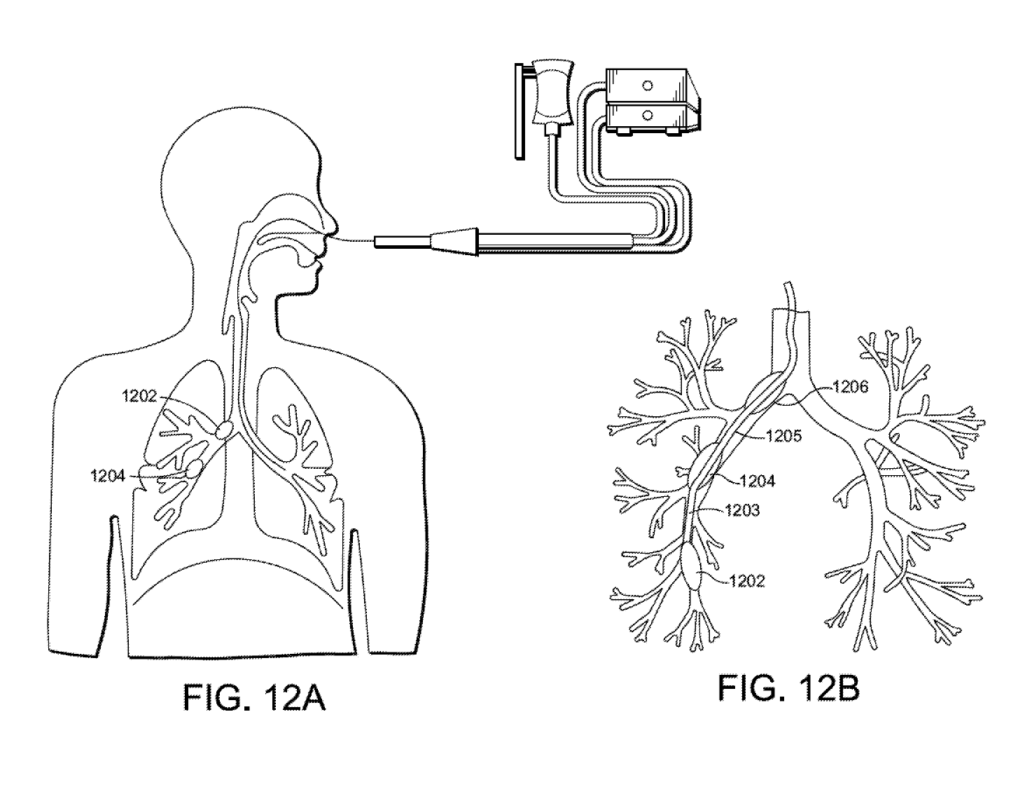

FIGS. 10A and 10B are diagrams showing isolation and target volume reduction for the treatment of jeopardized surfaces of the upper gastro-intestinal tract, according to one embodiment. For example, portions of the esophagus may be isolated, for subsequent treatment using LFUS, and/or other treatment modalities. As shown in FIGS. 10A and 10B, the volume to be treated may be bounded by one or more proximal volume-isolating structures 1002 and one or more distal volume-isolating structure 1004, 1006. The proximal and distal volume-isolating structures 1002, 1006 may be formed of any combination of balloons, sponges, or stents, for example. Other isolating structures may be utilized. For example, the stents may comprise a shape-memory metal, such as Nitinol®. According to a further embodiment shown in FIG. 10, relatively flat surfaces—such as surfaces within the stomach, may be effectively treated with LFUS or other treatment modalities through isolation of the affected area 1008 between one or more proximal volume-isolating structures 1010 and one or more distal volume-isolating structures 1014. The thus-isolated lesion 1008 may then be treated through the infusion of acoustically transmissive fluid and the generation of LFUS (and/or other forms of energy) by catheter or trocar 1012 of FIG. 10B within the isolated volume. Thereafter, as noted above, various other therapeutically-beneficial compounds may be administered.

FIG. 11 includes a diagram showing isolation and target volume reduction for the treatment of jeopardized surfaces of the lower gastro-intestinal tract, according to one embodiment. As shown, a steerable flexible catheter or trocar 1101 may be configured for insertion through the anus and into the large intestine. Such a device may comprise a plurality of volume-isolating structures that may comprise any combination of sponges, balloons, stents and/or any other structures configured to isolate a segment of the bowel. The balloons (such as shown at 1106 and 1104) may be configured to be selectably inflatable, deflatable and re-inflatable. Accordingly, the device may be configured to selectively be positioned for treatment in one area, the volume-isolating structures inflated and subsequently deflated and the device re-positioned to treat another area and the volume-isolating structures re-inflated to enable treatment at the new location. Alternatively, the LFUS delivering device may comprise a plurality of volume-isolating structures 1102, 1104, 1106, 1108 and may be configured to treat a plurality of segments simultaneously, by isolating more than one segment 1103, 1105, 1107 at a time. Vacuum, irrigation and evacuation structures may also be provided, as detailed above.

FIGS. 12A and 12B are diagrams showing isolation and target volume reduction for the treatment of jeopardized surfaces of the upper airway, according to one embodiment. Indeed, specific segments of the bronchial tree may be isolated using volume-isolating structures 1202, 1204 as shown in FIG. 12A. Another embodiment enables the isolation of several segments of the bronchi through the placement of volume-isolating structures 1202, 1204 and 1206 to create bounded volumes 1203 and 1205 within which fluids and LFUS may be delivered for treatment, as shown in FIG. 12B.

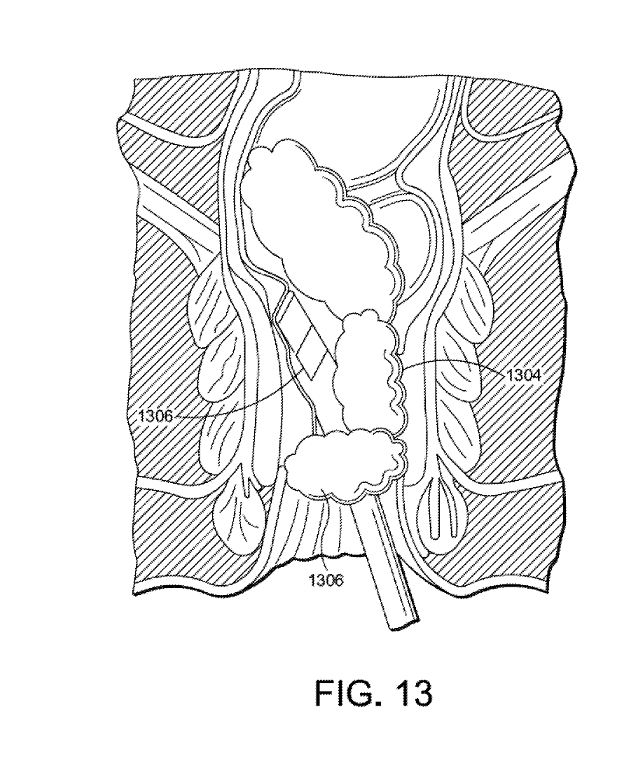

FIG. 13 includes diagrams showing isolation and target volume reduction for the treatment of jeopardized surfaces of the anorectal canal, according to one embodiment. As shown, one or more volume-isolating structures 1302, 1304, 1306 may create a bounded volume where none previously existed. This may be useful in treating a limited surface area of the lining of a natural passageway, such as the lower intestine, for example. As shown in FIG. 13, the volume-isolating structures 1302, 1304, 1306 may be in intimate contact with one another to create a bounded volume between them and the surface to be treated. According to one embodiment, one or more of the volume-isolating structures may comprise a polymeric sponge having, when fully swelled, a predetermined shape that is useful in isolating specific anatomical structures. Likewise, inflatable balloons may be configured to assume a specific shape when inflated and may, for example, comprise lumen enabling the introduction of an LFUS-generating device 1306 and/or fluid and/or gas evacuating structures, for example.

According to embodiments, isolation and/or reduction of the surface area and/or volume of treatment significantly increases LFUS efficacy via reduction of the acoustic energy dissipation and better delivery of the biologically active agents used for the treatment. Isolation and/or reduction of the surface area and/or volume of treatment also helps to decrease the amount of the biologically active agents and or their concentration that must be used to achieve the desired therapeutic effect. Moreover, in certain anatomical situations, it may be desirable to prevent the fluid(s) administered and/or LFUS or other energy applied from going into some other areas besides the treatment zone. Indeed, according to embodiments, one or more of the volume-isolating structures may comprise material(s) that are opaque to and/or poor conductors of acoustical energy (or of other energies applied) and/or create a generally fluid-tight barrier to prevent seepage of the delivered fluid to other, untargeted areas.

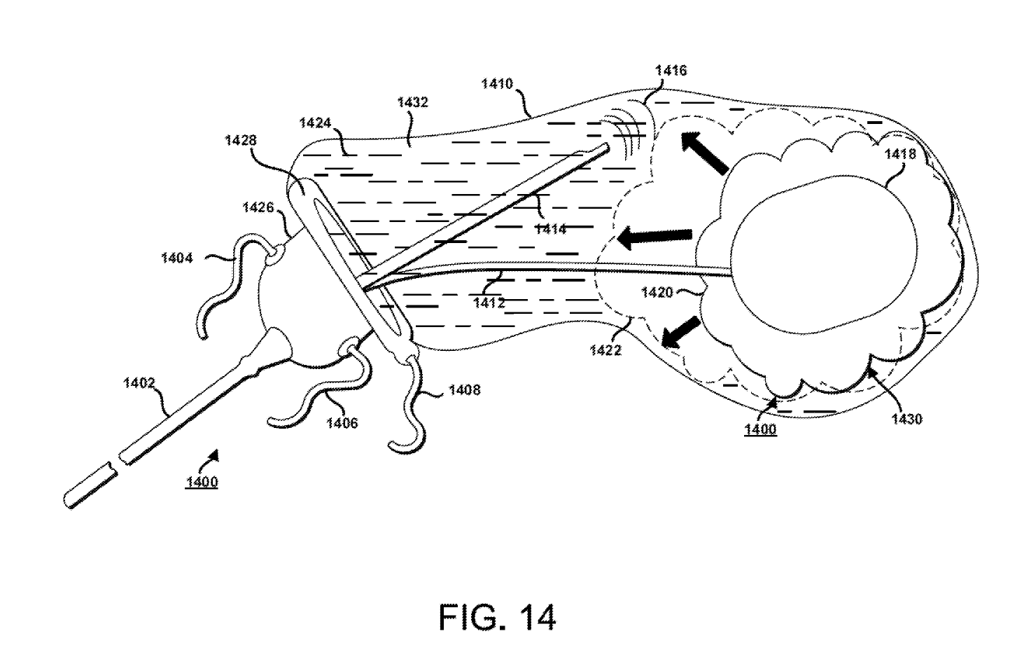

FIG. 14 shows a device according to one embodiment, deployed in an exemplary void within biological tissue. The device 1400, according to one embodiment, may comprise a proximal member 1426, an LFUS delivering device 1414 and a distal member 1430 connected to the proximal member via one or more conduits or flexible tube 1412. The tube 1412 may, according to embodiments, be a rigid tube made out plastic or metal/alloy comprising multiple lumens. Tube 1412 may, according to one embodiment, serve as an introducer for the proximal member 1426, inflation deflation port 1404, 1406 and/or may comprise a fluid injection lumen for introducing air of fluid into the distal member 1430. According to one embodiment, the tube 1412 may also function as a conduit into which the waveguide 1414 may be introduced into the bounded cavity 1410, particularly in the case in which the LFUS delivering device is to be placed into proximity of the distal member 1430. According to one embodiment, the proximal member may have, according to one embodiment, a funnel or dome shape, be inflatable and comprise a soft and/or inflatable or otherwise expandable annular lip 1428. The proximal member 1426 may comprise an elastomer, according to one embodiment. The annular lip 1428 may be configured to be inserted just inside the cavity to be treated, shown at 1432 in FIG. 14, and expanded so as to substantially seal the opening thereof. Indeed, the proximal member 1426 may be configured to adjust and accommodate to surface conformation and irregularities so as to produce a complete enclosure of the targeted space/tissue. The proximal member 1426 may be configured to enable access to the cavity 1432. Coupled to the proximal member 1426 through conduit 1412 is the distal member 1430. This coupling may be permanent or the distal member 1430 attached to the conduit 1412 may be configured to be move relative to the proximal member 1426 or the proximal member 1426 may be configured to move relative to the conduit 1412. For example, the distal member 1430 may be introduced first, and the proximal member 1426 may slide over the conduit 1412 into position.

According to one embodiment, the distal member 1430 may comprise, for example, an inner expandable member 1418 such as a balloon. The distal member may also comprise swellable material 1422. The swellable material 1422 may be configured to swell (expand in volume) when in the aqueous environment of the cavity 1432 and/or when exposed to a fluid 1424 introduced into the cavity 1432 through, for example, an irrigation port 1404 disposed within the proximal member 1426. Or, fluid may be delivered to distal member 1430 via conduit 1412, which may comprise a dual lumen, with the lumens being parallel, eccentric or concentric. The fluid 1424 may comprise, for example, an acoustically-transmissive fluid and/or a biologically active substance, such as an antibiotic, an antimicrobial agent, steroids, dyes, sono-sensitizers, photo-sensitizers, antiseptic preparations or other therapeutically beneficial fluids. The inner expandable member 1418 may be coupled to the proximal member 1426 through flexible tube 1412. The flexible tube 1412 may comprise a single or multiple lumens and extend through the proximal member 1426 and emerge therefrom within catheter or trocar 1402. The flexible tube 1412 may, for example, be configured to selectively deliver and evacuate air or a fluid to expand or collapse, respectively, the inner expandable member 1418. In the case in which the distal element 1430 is not inflatable or expandable through the introduction of fluids therein but is instead swellable through absorption of fluids within the cavity 1432, the flexible conduit need not comprise inner lumens. Indeed, swelling of the swellable layer 1420 may be achieved also by injecting fluid therein via one of the lumens in flexible tube 1412. According to one embodiment, however, the distal member 1430 may comprise both an expandable inner member 1418 such as a balloon and an outer layer of swellable material 1420. Fluids selectively introduced through the one or more lumen in the flexible tube 1412 may cause such a balloon to expand. The swellable material 1420 may comprise an antibiotic or other beneficial agent that may be released as the swellable materials absorbs fluid from the cavity 1432.

The expandable inner member 1418, according to one embodiment, may comprise an elastomeric balloon configured to occupy most of the volume within a closed cavity. Such balloon may be configured to conform to the inner wall of the closed cavity 1432, to collapse right onto the inner wall thereof, and/or may be configured to leave a thin rim of space between the balloon and the inner wall of the closed cavity 1432. According to embodiment, the expandable inner member 1418 may be filled with an acoustically-transmissive fluid, gel or mist to carry the LFUS. Indeed, the fluid, gel or mist may allow ultrasonic energy to travel from within the balloon via the fluid, gel or mist to the outer edge of the expandable inner member 1418, thereby enabling ultrasound to leave the expandable inner member 1418 to act directly on the inner surface of the tissue bounding the cavity 1432. Antimicrobials, or other beneficial agents may be introduced in the cavity 1432 and around the expandable inner member 1418. This allows the LFUS (however delivered), to act on the circulating liquid and to thus affect the inner surface of the organ's cavity 1432. Alternatively, the LFUS may travel through the bodily fluids or mucus naturally present in the cavity 1432.

The expandable inner member 1418 may be configured to be porous and elute fluid within the thin rim of free volume thereabout. Indeed, a liquid doped with various antimicrobials and other chemicals may be eluted from the expandable inner member 1418 to be circulated within this thin rim of space around the expandable inner member 1418. Alternatively, the expandable inner member 1418 may be configured to be impermeable. The fluid within the expandable inner member 1418 may be pre-heated prior to or after introduction within the expandable inner member 1418, to thereby enable the heated liquid to act on the inner wall of the organ's cavity 1432 and within the space between the expandable inner member 1418 and the surrounding tissue within the cavity 1432. According to one embodiment, the temperature and pressure of the liquid (which may be circulated) within the cavity 1432 may be monitored and controlled.

In use, the distal member 1430 may be introduced into a cavity or conduit defined by biological tissue in an un-inflated and un-swelled state, so as to facilitate insertion. Due to the aqueous environment (e.g., mucus, blood and/or other bodily fluids) within the cavity 1432, the swellable material 1422 may begin to absorb some of the fluid and expand. When the operator is satisfied that the distal portion 1430 is properly placed, the proximal portion 1426 may be inserted over the catheter 1402 or other guidewire-type structure and fitted to the opening (or a chosen site within the cavity or conduit). Air and/or fluids may be then be introduced through the catheter 1402 into the proximal member 1426 and/or the annular lip 1428. Fluid may also be introduced into flexible tube 1412 (configured, in this instance as a single or multiple lumen conduit), to thereby cause the inner expandable member 1418 to expand. The swellable material 1420 may swell and, either alone or in combination with expandable member 1418, expand to occupy a greater amount of space, as suggested by the dashed outline at 1422. The thus-expanded distal member 1430 may be utilized, according to embodiments, to plug an opening within a biological conduit and/or to reduce, through expansion thereof a free volume of the cavity or conduit 1432.

Indeed, as shown in FIG. 14, the distal member 1430, in expanding within the conduit or cavity 1432, reduces the otherwise free volume thereof. In turn, this reduced free volume tends to enhance the therapeutic activity of a number of treatment modalities. For example, substances may be delivered at a much greater concentration within the reduced free volume than would otherwise be advisable. According to one embodiment, an LFUS delivering device 1414 may be introduced into the cavity 1432 through catheter or trocar 1402 and through the proximal portion 1426. In this case, the reduced free volume of the cavity 1432 enables the LFUS energy delivered by LFUS delivering device 1414 to dissipate less than it otherwise would in a comparatively greater volume. Alternatively, the reduced free volume of the cavity 1432 may enable the LFUS to be delivered at a lower power (and thus with a smaller-sized LFUS delivering device 1414), as compared to the case wherein the cavity 1432 were comparatively larger. The efficacy of the LFUS energy may be increased by at least partially filling the cavity 1432 with an acoustically transmissive fluid 1424 that may also comprise biologically active substance, such as an antibiotic, an antimicrobial agent, steroids, dyes, sono-seusitizers, photo-sensitizers, antiseptic preparations and/or other therapeutically beneficial fluids. Fluid 1424 may be evacuated from the cavity 1432 via an evacuation port 1406.

According to embodiments, the liquid 1424 present and/or circulated within the cavity 1432 may be acted on by the generated LFUS. The beneficial action, on the targeted tissues, may be enhanced through the application of the LFUS, heat or light. For example, the active agents acted on by the liquid 1424 present and/or circulated within the cavity 1432 may comprise antibiotics, oxidizing agents (e.g., hydrogen peroxide or other peroxides), acidic agents, basic agents, ferric and ferrous salts and/or salts (e.g., Aluminum salts or other salts) that cause destruction of extracellular polysaccharide (EPS) substances that encase biofilm bacteria and/or that cause the detachment of EPS from a target surface. The liquid 1424 may also tend to produce nitrous oxide (NO) when exposed to ultrasound (LFUS or other) such as nitrous oxide synthetase within the tissue cells generated through ultrasound induction.

After the LFUS and/or other therapies have been applied to the tissue surfaces bounding the reduced free volume of the cavity 1424, the LFUS delivering device 1414 may be withdrawn through the catheter or trocar 1402 and the fluid 1424 within cavity 1432 may be drained through evacuation port 1406. Similarly, the expandable member 1418 may be deflated through evacuating air and/or the fluids therein through conduit 1412. The proximal member 1426 may also be deflated, as may be the annular lip 1428. The entire assembly 1400 may now be withdrawn from the cavity or conduit. Any entrance wound created to insert the device 1400 may be closed and/or the cavity may be packed or otherwise treated to complete the procedure. The cavity 1432, according to embodiments, may be a nasal cavity, a nasopharyngeal cavity, a portion of the upper or lower GI tract, airways, uterine and vaginal cavities, the anorectal canal and/or any other natural or constructed conduit or cavity within biological tissue.

It is to be understood that the structures shown in FIG. 14 are for illustrative and exemplary purposes only. It is also to be understood that the structures of the distal and proximal members 1430, 1426 may be adapted to the cavity or conduit into which they are to be deployed.

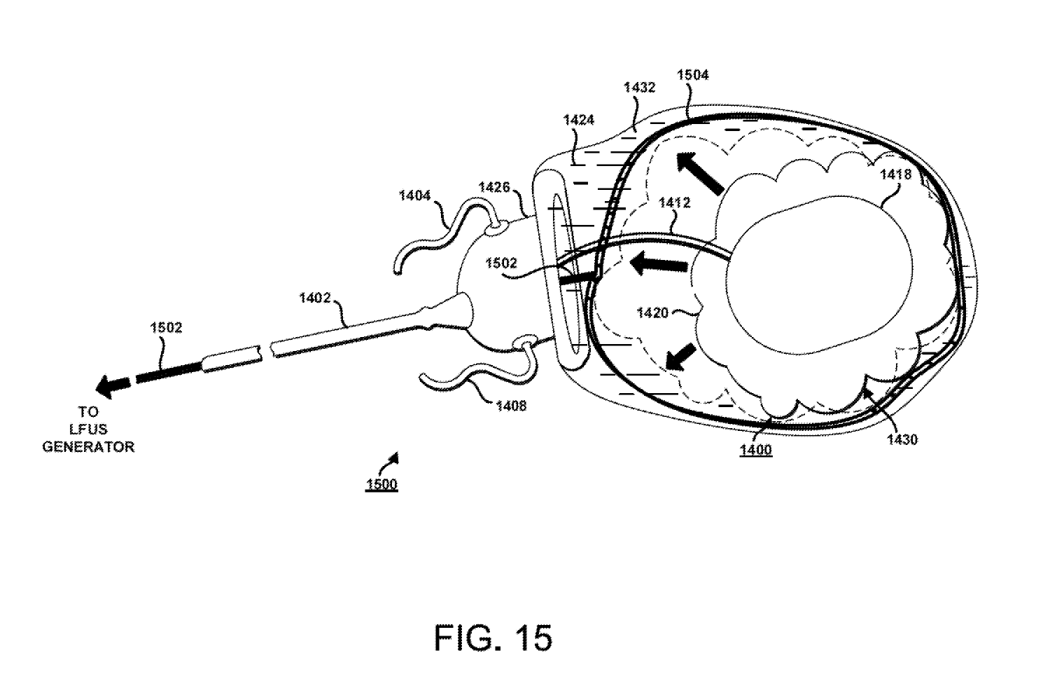

FIG. 15 shows a device according to a further embodiment, deployed in an exemplary cavity within biological tissue. The embodiment of the device 1500 as shown in FIG. 15 comprises an expandable distal member 1430 that is configured to be disposed within a biological cavity 1432 within biological tissue. The device 1500 may also comprise a catheter 1402, a flexible tube 1412 disposed within the catheter 1402 and extending to the distal member 1430. A proximal member 1426 may be provided, with the proximal member 1426 being configured to be inserted at least partially into the cavity 1432 over the flexible tube 1412 and to at least partially seal the cavity 1432. According to one embodiment, the catheter 1402 and the proximal member 1426 are configured to enable an ultrasound waveguide (wire 1502 is this embodiment) to be inserted in the catheter 1402 and through the proximal member 1426 to a position within the cavity 1432 between the proximal member 1426 and the distal member 1430. The structure and characteristics and functionality of elements numbered identically to corresponding structures shown in and described relative to FIG. 13 have been described above. Also shown in FIG. 15 is an expandable mesh 1502 that at least partially surrounds the expandable distal member 1430. According to one embodiment, the expandable mesh 1504 may be configured to conduct ultrasound energy (e.g., LFUS energy) applied thereto using the ultrasound wire 1502. According to one embodiment, therefore, the expandable mesh comprises an ultrasonically conductive material. For example, the expandable mesh 1504 may comprise a stainless steel mesh-like loose fabric that conducts LFUS energy applied thereto by ultrasonic wire 1502.

In use, the ultrasound wire 1502 induces the expandable mesh to vibrate at ultrasonic frequencies, which causes cavitation, micro-streaming and all phenomena discussed herein attendant to the application of LFUS to an aqueous medium such as fluid 1424. As the surrounding free volume within the cavity 1432 is greatly reduced by the presence of the expanded distal member 1430, the applied energy does not dissipate nearly as much as it otherwise would in a larger free volume. This enables the effect of the applied LFUS energy to targeted tissues to be greater than it otherwise would be in a comparatively larger volume or allows comparatively lower LFUS energies to be used to achieve the intended therapeutic effect. Indeed, the LFUS energy only need travel within the thin layer of fluid 1424 surrounding the expanded distal member 1430 and tends to lose less intensity than it would traveling a larger distance. According to one embodiment, the ultrasonic mesh 1504 may nebulize (i.e., vibrationally induce the atomization of) at least some of the fluid 1424 with which it comes into contact. Such nebulization may have beneficial action upon the targeted tissues, such as absorption of fluid 1424. It is to be noted that in this embodiment, the source of the LFUS energy is no longer a point source, as it is in the embodiment of FIG. 14.

The proximal member, the distal member and the balloon or balloons may comprise very pliable materials to fit and conform to irregularly-shaped cavities. For example, the constituent components of the devices and systems described and shown herein may be “custom made” for a particular cavity or conduit and/or for a particular person. For example, in an area such as the larynx, a mold can be made to specifically fit a defining region such as the laryngeal ventricle and stabilize a balloon such that the positioning is exact for precise delivery of treatment to a defined surface or volume. The precise fitting enable by such customization enables exposing targeted tissues to a predetermined treatment modality in a highly predictable manner.

According to embodiments, isolation (in the nasopharyngeal cavity, for example) may be achieved through deployment of various expandable mechanisms such as balloons, swellable sponges, expandable stents and/or combinations thereof. The expandable volume-isolating structures may have multiple functionalities, such as to isolate the treatment surface(s), as well as to serve as inlets and/or outlet for the circulating fluids (both acoustic fluid as well as therapeutically-beneficial fluids such as antibiotic fluids and the like. According to embodiments, isolation and treatment of the jeopardized surface(s) may be carried out on the various tubular, spherical, semi spherical and cavernousal areas of the mammal body. Examples of conditions and structures that may be treated according to embodiments, include, for example, sinusitis, bronchitis, middle ear and mastoid, stomach, gall bladder, colon, urinary bladder, interior cavity of kidney, urethra and prostate, blood vessels both arterial and venous, atrium and ventricle of heart, brain abscess, osteomyelitis, any soft tissue abscess cavity, the mouth, and interior cavities of the uterus and vagina.

As noted above, isolation and LFUS (or other) treatment may be combined with a number of the modern interventional modalities including, for example, endoscopy, colonoscopy and bronchoscopy. According to embodiments, a treatment device may be configured to isolate a proposed treatment region (jeopardized surface) by, for example, capping (enclosing a surface), by trapping (closing ingress and egress from a space) or by reducing the treatment volume of a large space to just over the affected or jeopardized surface, thereby enabling maximal effectiveness of both the applied energy (whether LFUS or other) and any delivered therapeutic fluids or materials. Such a treatment device may be configured to deliver an ultrasound transmissive (conducting) medium to all surfaces of isolated space, surface or volume. Energy may then be applied to treat the target region by, for example, mechanically and/or chemically removing planktonic and biofilm bacteria from the surface through a micro-bubble scrubbing using applied ultrasonic energy within a bounded volume. The treatment device may be configured, according to embodiments, to deposit biologically active agents such as, without limitation, antibacterial, antifungal, antiviral agents. For example, antibiotics, enzymes, surfactants, reactive oxygen species, reactive nitrogen species and/or others may be delivered and evacuated from the bounded surface, area or volume. The local production of nitric oxide (NO) may be induced. Thereafter, regenerative organisms may be delivered to the bounded volume to reconstitute the native or a replacement microbiome. The present device may also comprise structures effective to remove detritus from the area, surface or volume under treatment. According to embodiments, any surface of the body comprising an infection or necrotic tissue may be treated, whether internal or external, in an early (acute) or late (chronic) stage of development.

While certain embodiments of the disclosure have been described, these embodiments have been presented by way of example only, and are not intended to limit the scope of the disclosure. Indeed, the novel methods, devices and systems described herein may be embodied in a variety of other forms. Furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure. For example, those skilled in the art will appreciate that in various embodiments, the actual physical and logical structures may differ from those shown in the figures. Depending on the embodiment, certain steps described in the example above may be removed, others may be added. Also, the features and attributes of the specific embodiments disclosed above may be combined in different ways to form additional embodiments, all of which fall within the scope of the present disclosure. Although the present disclosure provides certain preferred embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

1. A method, comprising:
isolating at least a portion of a volume of a passageway or cavity within tissue using at least a first volume-isolating structure;
introducing a fluid within the isolated volume of the passageway or cavity;
producing a therapeutic effect on a surface of the tissue within the isolated volume of the passageway or cavity;
evacuating the fluid from the isolated volume of the passageway or cavity; and
removing at least the first volume-isolating structure.

2. The method of claim 1, wherein at least the first volume-isolating structure comprises a balloon; wherein isolating comprises inserting the balloon within the passageway or cavity; and wherein removing comprises deflating the balloon.

3. The method of claim 1, wherein the isolated volume of the passageway or cavity comprises an interstitial space between the surface of the tissue and a surface of the at least first volume-isolating structure.

4. The method of claim 1, wherein isolating further comprises using a second volume-isolating structure within the passageway or cavity and wherein introducing comprises introducing the fluid within the cavity or passageway between the first and the second volume isolating structures.

5. The method of claim 4, wherein at least one of the first volume-isolating structures comprises at least one of a balloon, a swellable sponge, a plug and a stent.

6. The method of claim 5, wherein at least one of the first volume-isolating structures is configured to conform to a shape of an opening.

7. The method of claim 1, wherein producing a therapeutic effect comprises applying Low frequency Ultra Sound (LFUS) within the isolated volume of the passageway or cavity.

8. The method of claim 7, wherein the LFUS is emitted from one of an ultrasonically functional catheter and a waveguide.

9. The method of claim 7, further comprising introducing a waveguide within the isolated volume of the passageway or cavity, the waveguide comprising a housing comprising a plurality of openings along a length thereof and wherein the LFUS is emitted from the plurality of openings to cause the cavitation within the introduced fluid.

10. The method of claim 1, wherein isolating at least the portion of the volume of the passageway or cavity is carried out within soft tissue and wherein the passageway or cavity is created from an excision of the soft tissue and naturally-occurring.

11. The method of claim 1, wherein isolating at least the portion of the volume of the passageway or cavity is carried out within bone and wherein the passageway or cavity is created from an excision of the soft tissue and naturally-occurring.

12. The method of claim 1, further comprising providing a therapeutic agent to the treated surface of the tissue.

13. The method of claim 1, wherein introducing comprises introducing an acoustically-transmissive fluid within the isolated volume of the passageway or cavity.

14. The method of claim 1, wherein introducing comprises introducing an acoustically-transmissive fluid in the form of a mist within the isolated volume of the passageway or cavity.

15. The method of claim 1, wherein introducing introduces a fluid that is doped with a therapeutically-beneficial agent.

16. The method of claim 15, wherein the therapeutically-beneficial agent comprises at least one of an antibiotic agent, an antiviral agent, an antifungal agent, an enzyme, a surfactant, a reactive oxygen specie, a reactive nitrogen specie, a steroid and an immune stimulating agent, a biologic product and a radioactive brachytherapy agent.

17. The method of claim 1, wherein treating a surface of the tissue comprises introducing a catheter or a trocar, comprising a plurality of openings along a length thereof, within the isolated volume of the passageway or cavity and introducing a low frequency ultrasound (LFUS) waveguide into the catheter or trocar such that LFUS energy propagates into the isolated volume of the passageway or cavity through the openings in the catheter or trocar.

18. The method of claim 1, wherein treating a surface of the tissue comprises disposing a low frequency ultrasound (LFUS) waveguide in or against the first volume-isolating structure.

19. The method of claim 1, wherein at least the first volume-isolating structure is configured to be acoustically transmissive.

20. The method of claim 1, wherein producing a therapeutic effect comprises causing cavitation within the introduced fluid.

21. The method of claim 1, wherein producing a therapeutic effect comprises causing one of streaming and micro-streaming within the introduced fluid.

22. The method of claim 1, wherein producing a therapeutic effect comprises causing sonophoresis of a therapeutically beneficial agent.

23. A method, comprising:
disposing an expandable member within a void at least partially bounded by biological tissue;
expanding the expandable member to occupy most of a free volume of the void bounded by the biological tissue such as to leave a decreased free volume between an outer surface of the expandable member and the biological tissue bounding the void;
within the decreased free volume, producing a therapeutic effect on at least a portion of the biological tissue; and
at least partially collapsing and removing the expandable member from the void.

24. The method of claim 23, wherein the expandable member comprises an expandable balloon.

25. The method of claim 24, further comprising at least partially filling the expandable balloon with a fluid.

26. The method of claim 25, wherein the fluid comprises at least one of an acoustically transmissive fluid, an antibiotic agent, an antiviral agent, an antifungal agent, an enzyme, a surfactant, a reactive oxygen specie, a reactive nitrogen specie, a steroid and an immune stimulating agent.

27. The method of claim 25, further comprising introducing a source of Low Frequency Ultrasound (LFUS) into the fluid within the expandable balloon.

28. The method of claim 23, wherein the expandable member comprises a swellable material.

29. The method of claim 23, further comprising introducing a fluid into the decreased free volume between the outer surface of the expandable member and the biological tissue bounding the void.

30. The method of claim 23, further comprising introducing Low Frequency Ultrasound (LFUS) into the decreased free volume between the outer surface of the expandable member and the biological tissue bounding the void.

31. A device, comprising:
an expandable distal member configured to be disposed within a biological conduit or cavity within biological tissue;
a catheter;
a flexible tube disposed within the catheter and extending to the distal member;
a proximal member, the proximal member being configured to be inserted at least partially into the cavity over the flexible tube and to at least partially seal the biological conduit or cavity,
wherein the catheter and the proximal member are configured to enable an ultrasound waveguide or wire to be inserted in the catheter and through the proximal member to a position within the biological conduit or cavity between the proximal member and the distal member.

32. The device of claim 31, wherein the distal member is configured to absorb fluid within the cavity and to swell.

33. The device of claim 31, wherein the distal member is configured to absorb fluid and to swell.

34. The device of claim 31, wherein the distal member is configured to expand and occupy most of the cavity.

35. The device of claim 31, wherein the flexible tube comprises a balloon that is inflatable through introduction of at least one of air and a fluid delivered through a lumen in the flexible tube.

36. The device of claim 35, further comprising a layer of swellable material disposed on an outer surface of the balloon.

37. The device of claim 31, wherein the proximal member further comprises a lip configured to substantially seal the biological conduit or cavity.

38. The device of claim 37, wherein the lip is configured to be expandable.

39. The device of claim 31, wherein at least the proximal member comprises an elastomer.

40. The device of claim 31, wherein the catheter and the proximal member are configured to deliver a fluid into the biological conduit or cavity.

41. The device of claim 31, wherein the proximal member comprises an irrigation port and an evacuation port, configured to introduce fluid into and evacuate fluid from the sealed biological conduit or cavity.

42. The device of claim 31, further comprising an expandable mesh at least partially surrounding the expandable distal member, the expandable mesh being configured to conduct ultrasound energy applied using the ultrasound waveguide.

43. The device of claim 42, wherein the ultrasound waveguide comprises a wire configured to couple to the expandable mesh.

44. The device of claim 31, custom-made for a particular patient, to enable exposing targeted tissues to a predetermined treatment modality in a predictable manner.
